(19) United States
(12) Patent Application Publication
(10) Pub. No.: US 2016/0357935 A1
(43) Pub. Date: Dec. 8, 2016

(54) NOVEL SET OF FASTING BLOOD BIOMARKERS TO DETECT PATIENTS WITH IMPAIRED GLUCOSE TOLERANCE

(71) Applicant: True Health Diagnostics LLC, Frisco, TX (US)

(72) Inventors: James V. Pottala, Sioux Falls, SD (US); Steve Varvel, Richmond, VA (US)

(21) Appl. No.: 15/171,213

(22) Filed: Jun. 2, 2016

Related U.S. Application Data
(60) Provisional application No. 62/171,093, filed on Jun. 4, 2015.
(60) Provisional application No. 62/180,346, filed on Jun. 16, 2015.

(51) Int. Cl. G06F 19/00; G01N 33/50; C12Q 1/28; G01N 33/92; G01N 33/68

(52) U.S. Cl. CPC G06F 19/3431; G01N 33/92; G01N 33/6893; C12Q 1/28; G01N 33/50; C12Y 111/02002; G01N 2800/042; G01N 2800/50; G01N 2800/52; G01N 2333/908

(57) ABSTRACT

The present disclosure relates to methods of predicting the likelihood of a subject having impaired glucose tolerance or insulin resistance by measuring a novel set of fasting blood biomarkers.

### PRIORITY CLAIM

This application claims priority to U.S. Application No. 62/171,093, filed Jun. 4, 2015, and U.S. Application No. 62/180,346, filed Jun. 16, 2015, the entire contents of which are incorporated herein by reference.

### FIELD

The invention relates to the field of clinical diagnostics. More specifically, the invention involves clinical testing of biomarkers to predict the likelihood of a subject having impaired glucose tolerance or insulin resistance.

### BACKGROUND

Currently, a number of tests exist that can diagnose whether a patient has a normal glucose tolerance (NGT) or an impaired glucose tolerance (IGT). One test is to measure the blood glucose level at a 2-hour point of oral glucose tolerance test (OGTT): elevated blood glucose above 140 mg/dL indicates abnormal glucose tolerance. Other tests that may indicate abnormal glucose tolerance include measuring levels of fasting blood glucose, insulin, pro-insulin, c-peptide, HbAlC, fructosamine, glycation gap, 1,5-Anhydroglucitol (1,5 AG), “clamp-like index” (CLIX) scoring (an index obtained from plasma OGTT glucose and C-peptide levels and serum creatinine), homeostasis model assessment-estimated insulin resistance (HOMA IR) scoring, and immuno reactive insulin (IRI) scores based on combinations of alpha hydroxybutyrate (AHB), linoleoyl-GPC (L-GPC), and oleic acid weighted by insulin or body mass index (BMI). The above tests, used alone or in combination, can detect the presence of pre-diabetes (metabolic syndrome) and early insulin resistance in patients who are normoglycemic in fasting state.

The best current predictors of fasting normoglycemic patients who may be at risk of developing diabetes are OGTTs and CLIX scoring of OGTTs. Both techniques involve testing multiple analytes at multiple time-points, requiring the patient to have a blood sample drawn at baseline (fasting) and to drink a beverage containing a known quantity of glucose, and subsequently contacting patient blood samples and measuring the levels of various analytes (e.g. glucose, insulin, pro-insulin, c-peptide, creatinine) at fasting baseline and at various time intervals after dosing with the glucose load. Most OGTTs and CLIX scoring require a patient to remain in the doctor's office for 2 hours post dose, and most clinicians only test baseline samples and compare to the testing at the 2 hour time point, not the labor-intensive additional blood draws for 3-5 times during the 2-hour period necessary for the CLIX scoring, due to labor and cost constraints. Moreover, complicated and laborious mathematical calculations need to be performed in order to optimize detection of at-risk individuals with these techniques. Additionally, kidney function (approximated by blood creatinine levels/estimated Glomerular Filtration Rate (eGFR)) needs to be accounted for with these techniques, requiring a further step.

Elevated 2-hr plasma glucose (140-199 mg/dL) during a 75 gm OGTT, also known as IGT, is a defining feature of prediabetes¹; it also identifies individuals at increased risk for diabetes, cardiovascular disease, and other complications.^{2,3} Studies have shown that incident diabetes can be reduced by 40-60% with lifestyle or 30-70% with pharmacologic interventions in patients with IGT.⁴⁻⁸ Prediabetes affects more than 1 out of 3 American adults,⁹ an epidemic that necessitates urgent implementation of improved primary prevention and early treatment strategies.

Measurement of fasting plasma glucose (FPG) and hemoglobin-Alc (HbAlc) are widely used for detecting individuals with prediabetes. However, when used alone, FPG and HbAlc fail to detect up to 30% of individuals with IGT, and there is discordance among these tests.¹⁰

Routine use of OGTT in screening for prediabetes in primary care settings would be ideal; however, OGTT has historically been underutilized due to time, cost, and inconvenience for both patients and medical practitioners in busy practices.

Hence, there is a need in the art more clinically pragmatic screening tools are needed for diagnostic biomarkers and tests that can identify patients at risk of developing Type 2 diabetes, the risk of disease progression in patients with insulin resistance, and especially to identify IGT in the primary care setting.

### SUMMARY

The methods disclosed herein for predicting likelihood of a subject having abnormal glucose tolerance may comprise: obtaining, from a biological sample collected from a subject, measured levels of a combination of biomarkers comprising C-peptide, myeloperoxidase (MPO), and high-density lipoprotein (HDL) cholesterol (HDL-C); and calculating an index score based on the measured levels of the biomarkers, wherein the index score involves a mathematical transformation; wherein an elevated index score correlates with an increased likelihood of elevation of blood glucose to ≧140 mg/dL at 2 hours after oral glucose tolerance test and indicates that the subject has an increased likelihood of having abnormal glucose tolerance, and wherein a low index score correlates with a decreased likelihood of elevation of blood glucose to ≧140 mg/dL at 2 hours post oral glucose tolerance test and indicates that the subject has a decreased likelihood of having abnormal glucose tolerance.

In some embodiments, the likelihood can be predicted by a single measurement of a single biological sample collected from a fasting subject. In other embodiments, the likelihood can be predicted by a single measurement of a single biological sample collected from a non-fasting subject.

In some embodiments, the index score replaces the oral glucose tolerance test (OGTT) to predict likelihood of the subject having abnormal glucose tolerance.

In some embodiments, the biomarkers in step (a) additionally comprise alpha hydroxybutyrate (AHB).

In some embodiments, the biomarkers in step (a) additionally comprise at least one biomarker relating to adipose tissue insulin resistance, and wherein the biomarker relating to adipose tissue insulin resistance is a total free fatty acid or a component fatty acid species of a total free fatty acid.

In some embodiments, the biomarkers in step (a) additionally comprise at least one biomarker relating to pancreatic beta cell dysfunction and/or exhaustion, and wherein the biomarker relating to pancreatic beta cell dysfunction and/or exhaustion is selected from the group consisting of intact pro-insulin, and a fragment of any form of insulin.

In some embodiments, the biomarkers in step (a) additionally comprise at least one biomarker relating to adipokine function, and wherein the biomarker relating to adipokine function is selected from the group consisting of adiponectin, leptin, tumor necrosis factor alpha (TNFα), resistin, visfatin, dipeptidyl peptidase-4 (DPP-IV), omentin, and apelin

In some embodiments, the biomarkers in step (a) additionally comprise at least one biomarker relating to functional enhancement of insulin secretion by beta cells, and wherein the biomarker relating to functional enhancement of insulin secretion by beta cells is selected from the group consisting of linoleoyl-glycerophosphocholine (L-GPC), an incretin, arginine, and other biological secretagogues and potentiators.

In some embodiments, the biomarkers in step (a) additionally comprise at least one biomarker relating to inhibition of beta cell function, and wherein the biomarker relating to inhibition of beta cell function is selected from the group consisting of glutamate, gamma-Aminobutyric acid (GABA), and other biomarkers with demonstrated beta cell toxicity or suppression of insulin secretion in response to glucose stimulation.

In some embodiments, the biomarkers in step (a) additionally comprise at least one biomarker relating to muscle and/or hepatic insulin resistance, and wherein the biomarker relating to muscle and/or hepatic insulin resistance is selected from the group consisting of ferritin, iron saturation, acyl-carnitine, carnitine, creatine, and a branched-chain amino acid.

In some embodiments, the biomarkers in step (a) additionally comprise at least one biomarker relating to total glycemic control, and wherein the biomarker relating to total glycemic control is selected from the group consisting of glucose, HbAlc, fructosamine, glycation gap, D-mannose, and 1,5-anhydroglucitol (1,5-AG).

In some embodiments, the biomarkers in step (a) additionally comprise at least one biomarker relating to inflammation control, and wherein the biomarker relating to inflammation control is selected from the group consisting of lipoprotein-associated phospholipase A2 (LpPLA2), fibrinogen, high sensitivity C-reactive protein (hsCRP), F2-isoprostanes, serum amyloid A and variants thereof, HSP-70, IL-6, TNF-α, haptoglobin and variants thereof, secretory phospholipase A2 (sPLA2), pregnancy-associated plasma protein-A (PAPP-A), and mannose binding lectin (MBL) level, activity, genetic polymorphisms or known haplotypes thereof.

In some embodiments, the mathematical transformation comprises: i) multiplying the measured level of each of the biomarkers by a pre-determined exponent to create a product of exponentiation for each of the biomarkers; ii) multiplying the product of the exponentiation for each of the biomarkers generated from step i) other than the product of exponentiation generated from HDLC to form a multiplied product of the exponentiations; iii) dividing the multiplied product of the exponentiations generated from step (ii) by the product of exponentiation generated from HDLC to generate a divided product; and iv) logarithmically transforming the divided product generated from step iii).

In some embodiments, the pre-determined exponent for each biomarker is derived from values within the 90% confidence interval of the biomarker measurement distribution in a population study. In other embodiments, the pre-determined exponent for each biomarker is the median or mean from values within the 99% confidence interval of the biomarker measurement distribution in the population study.

In some embodiments, the index score comprises a calculation of the form:

$$LN [ \frac{{Cpeptide}^{a} * {AHB}^{b} * {MPO}^{c}}{{HDLC}^{d}} ]$$

In other embodiments, the index score comprises a calculation of the form:

$$- 0.7 + LN [ \frac{{Cpeptide}^{1.4} * {AHB}^{1.5} * {MPO}^{0.8}}{{HDLC}^{d 2.1}} ]$$

In other embodiments, the index score comprises a calculation of the form:

$$LN [ \frac{{Cpeptide}^{a} * {FFA}^{b} * {MPO}^{c}}{{HDLC}^{d}} ]$$

In other embodiments, the index score comprises a calculation of the form:

$$2.9 + LN [ \frac{{Cpeptide}^{1.5} * {FFA}^{0.9} * {MPO}^{0.7}}{{HDLC}^{2.2}} ]$$

In some embodiments, the methods further comprise: obtaining values for one or more base model factors to predict the likelihood of the subject having abnormal glucose tolerance; calculating a based model score for the subject based on one or more values of the base model factors; and combining the index score obtained from step b) of claim 1 with the calculated base model score, wherein the combined score is compared to reference values from a population.

In some embodiments, the based model factor is fasting glucose, and wherein the base model score is calculated by logarithmically transforming a measured level of fasting glucose in the subject and multiplied by a weighting factor (beta).

In some embodiments, the combining the index score obtained from step b) of claim 1 with the base model score provides an improved likelihood prediction than the base model score alone.

In some embodiments, the combining the index score obtained from step b) of claim 1 with the base model score causes a net reclassification improvement (NRI) of the subject from having normal glucose tolerance (NGT) to impaired glucose tolerance (IGT), or from having IGT to NGT

In some embodiments, the NRI is at least about 10%.

In some embodiments, the methods further comprise administering a therapy regimen for the treatment or prevention of abnormal glucose tolerance.

In some embodiments, the methods further comprise monitoring the levels of the biomarkers in the subject to assess progression, improvement, normalization, and/or treatment efficacy, wherein the monitoring step comprises repeating steps a)-c) based on the levels of the biomarkers from a biological sample in the subject obtained at a later time.

A novel abnormal glucose tolerance risk score is described, based on measurements of a small subset of fasting biomarkers related to cardio-metabolic risk, strongly distinguished individuals with high probability for IGT, based on 2-hr 75 g OGTT.

The risk score also indicates IGT specifically.

The best-fitting and most parsimonious model included C-peptide, AHB, MPO, and HDL-C.

The an alternative model included C-peptide, FFA, MPO, and HDL-C.

This novel blood biomarker risk score can be used by busy clinicians in order to efficiently address and intervene on IGT and risk for disease progression.

### DESCRIPTION OF THE FIGURES

FIG. 1 depicts classification of study participates in the validation cohort having a 2-hour glucose ≧140 mg/dL and demonstrates that there was a significant increase in the area under the ROC curve when modified risk score (IGT-RS) was added to base model score (age, gender, body mass index (BMI), and fasting glucose.

FIG. 2 demonstrates linear trends were significant (all p<0.0001) across quartiles when the modified IGT risk score was stratified by quartiles for all participants and then the IGT prevalence rates were tested within the Health Diagnostic Laboratory (HDL) and Insulin Resistance Atherosclerosis Study (IRAS) cohorts. All neighboring quartiles were significantly different (p,0.05), unless otherwise noted as non-significant (NS).

FIG. 3 demonstrates IGT prevalence by increasing quartiles as assessed by three different modified index score models.

FIG. 4A corresponds to the models FIG. 3 and depicts classification of study participates in the training cohort having a 2-hour glucose ≧140 mg/dL and demonstrates that there was a significant increase in the area under the ROC curve when modified risk score (New Fasting) was added to base model score (age, gender, body mass index (BMI), and fasting glucose. FIG. 4B depicts a similar significant increase in the area under the ROC curve when modified risk score (New Fasting) was added to base model score (age, gender, body mass index (BMI), and fasting glucose in the validation cohort.

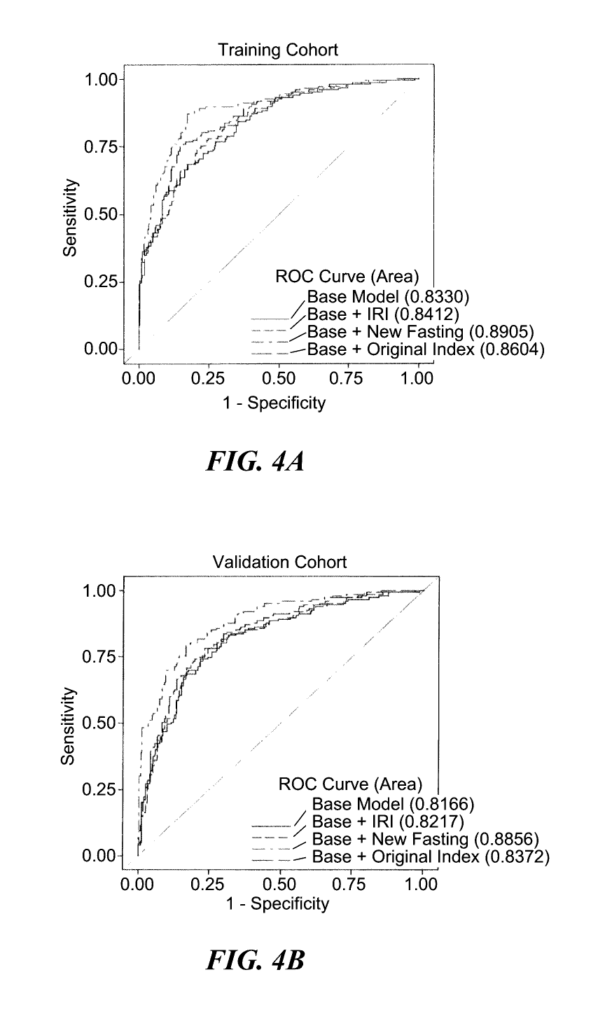

FIG. 5 demonstrates results from fasting sensitivity analysis.

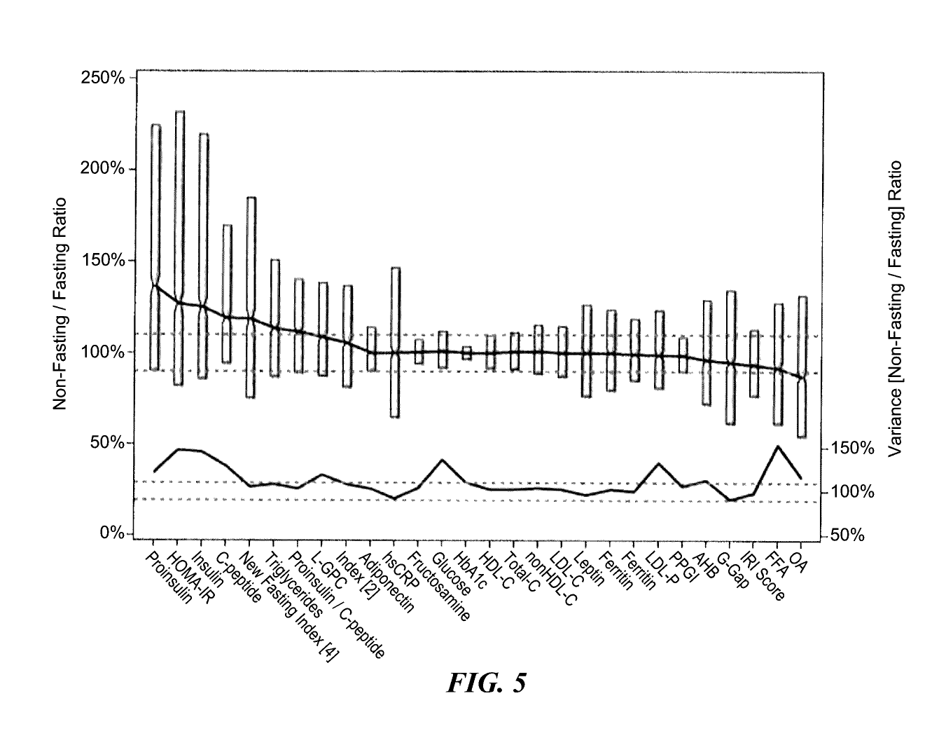

FIG. 6A depicts cohort sensitivity for Original Non-Fasting Index. FIG. 6B depicts cohort sensitivity analysis for New Fasting Index modified risk score.

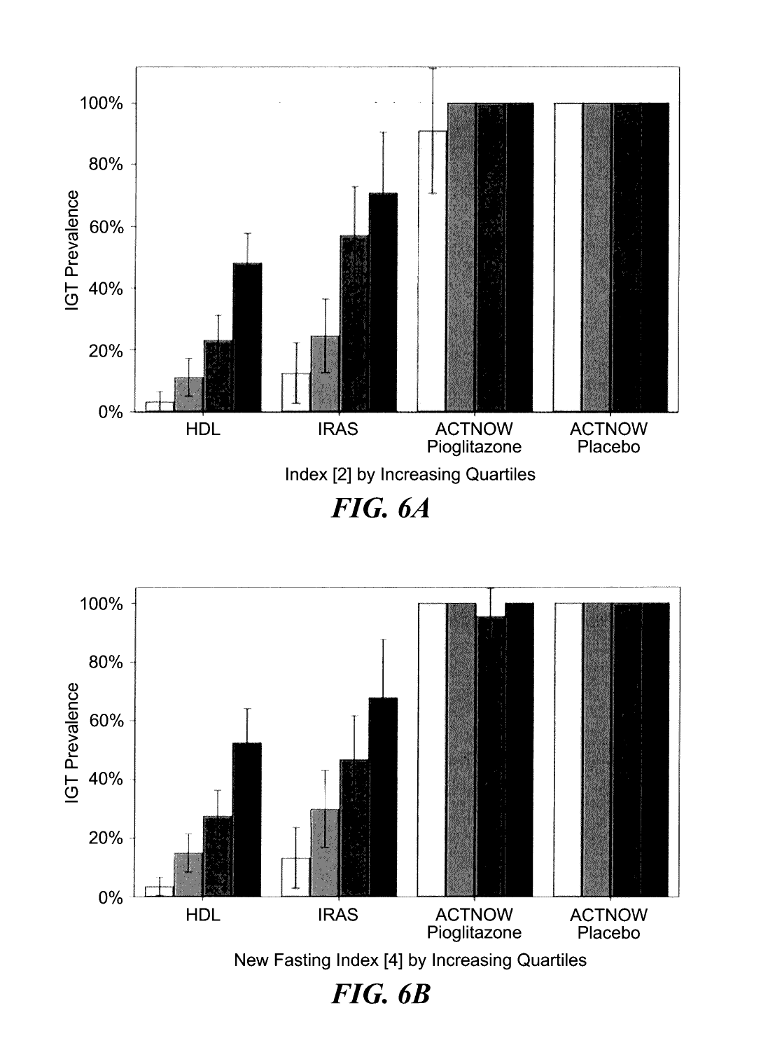

### DETAILED DESCRIPTION

This disclosure is predicated on the discovery of novel abnormal glucose tolerance risk scores based on measurements of a small subset of fasting biomarkers related to cardio-metabolic risk and which strongly distinguished individuals with high probability for IGT, based a conventional oral glucose tolerance test (OGTT).

One aspect of the disclosure relates to a method for predicting likelihood of a subject having abnormal glucose tolerance. The method comprises: a) obtaining, from a biological sample in a subject, measured level of a combination of biomarkers relating to at least three of the following physiological processes: adipose tissue insulin resistance, pancreatic beta cell dysfunction and/or exhaustion, muscle and/or hepatic insulin resistance, functional enhancement of insulin secretion by beta cells, inhibition of beta cell function, adipokine function, total glycemic control, and inflammation control; b) calculating a score based on the measured levels of the biomarkers, wherein the score calculation involves a mathematical transformation, and c) comparing the score to reference values from a population. An elevated score correlates with an increased likelihood of elevation of blood glucose to ≧140 mg/dL at 2 hours after oral glucose tolerance test and indicates that the subject has an increased likelihood of having abnormal glucose tolerance. A low score correlates with a decreased likelihood of elevation of blood glucose to ≧140 mg/dL at 2 hours post oral glucose tolerance test and indicates that the subject has a decreased likelihood of having abnormal glucose tolerance.

Another aspect of the disclosure relates to a method of determining abnormal glucose tolerance in a subject. The method comprises predicting a glucose disposal rate in a subject by analyzing a biological sample from a subject to determine a level(s) of biomarkers relating to three or more physiological processes in the sample. The three or more physiological processes are selected from the group consisting of: adipose tissue insulin resistance, pancreatic beta cell dysfunction and/or exhaustion, muscle and/or hepatic insulin resistance, functional enhancement of insulin secretion by beta cells, inhibition of beta cell function, adipokine function, total glycemic control, and inflammation control. The level(s) of the biomarkers in the sample is compared to glucose disposal reference levels of the biomarkers to determine insulin sensitivity in the subject.

Another aspect of the disclosure relates to a method of determining abnormal glucose tolerance in a subject. The method comprises predicting a glucose disposal rate in a subject by analyzing a biological sample from a subject to determine a level(s) of three or more biomarkers chosen from the group consisting of: total free fatty acids, ferritin, c-peptide, AHB, L-GPC, HDLC, and adiponectin.

Another aspect of the disclosure relates to a method of determining abnormal glucose tolerance in a subject. The method comprises predicting a glucose disposal rate in a subject by analyzing a biological sample from a subject to determine a level(s) of three or more biomarkers comprising at least total free fatty acids, c-peptide, and adiponectin.

The method can involve multiple steps such as patient sampling, laboratory analysis, and mathematical transformation of data, so that appropriate therapeutic steps can be taken.

A biological sample can be obtained from a living subject to analyze the biomarkers. Suitable biological samples include, but are not limited to, human biological matrices, blood, plasma, serum, urine, saliva, synovial fluid, ascitic fluid, or other biological tissue or fluid. For example, the sample may be fresh blood or stored blood or blood fractions. The sample may be a blood sample expressly obtained for the assays of this invention or a blood sample obtained for another purpose which can be sub sampled for use in accordance with the methods described herein. For instance, the biological sample may be whole blood. Whole blood may be obtained from the subject using standard clinical procedures. The biological sample may be a blood sample separated out into plasma or serum for analysis. Plasma may be obtained from whole blood samples by centrifugation of anti-coagulated blood.

The biological sample can be measured and analyzed on any devices or combined devices known to one skilled in the art capable of detecting and quantifying amounts of organic molecules, biological metabolites, and/or proteins in the sample, using any method known to one skilled in the art. For example, a mass spectrometer may be used independently or in conjunction with a liquid or gas chromatography instrument or ion mobility spectrometer to analyze the biomarkers in the sample. Alternative instruments used in the sample analysis may include NMR, or devices for immunological detection.

The measurement of levels of the biomarkers in the sample can be carried out with or without sample preparation. The sample may be pretreated as necessary by dilution in an appropriate buffer solution, concentrated if desired, or fractionation or separation of biomarkers from each other by any number of methods including but not limited to ultracentrifugation, chromatography, fractionation by fast performance liquid chromatography (FPLC), or precipitation. Any of a number of standard aqueous buffer solutions, employing one of a variety of buffers, such as phosphate, Tris, or the like, at physiological to alkaline pH can be used.

Measurements of the biomarkers in the sample can be carried out using any suitable devices to measure, among other values, the concentrations, levels of activity, or absolute amounts of the biomarkers. Thus, the terms “quantities,” “levels,” “amounts,” “concentrations,” and “levels of activity,” when used to describe the amount of biomarkers, are herein interchangeable.

The biomarkers measured relate to the following physiological processes: 1) adipose tissue insulin resistance, 2) pancreatic beta cell dysfunction and/or exhaustion, 3) muscle and/or hepatic insulin resistance, 4) functional enhancement of insulin secretion by beta cells, 5) inhibition of beta cell function, 6) an adipokine, 7) total glycemic control, and 8) inflammation control.

The biomarkers used may relate to adipose tissue insulin resistance. Suitable biomarkers include, but are not limited to, a total free fatty acid or a component fatty acid species of a total free fatty acid.

The biomarkers used may relate to pancreatic beta cell dysfunction and/or exhaustion. Suitable biomarkers include, but are not limited to, c-peptide, intact pro-insulin, and a fragment of any form of insulin.

The biomarkers used may relate to adipokine function. Suitable biomarkers include, but are not limited to, adiponectin, leptin, TNFα, resistin, visfatin, DPP-IV, omentin, and apelin.

The biomarkers used may relate to functional enhancement of insulin secretion by beta cells. Suitable biomarkers include, but are not limited to, L-GPC, an incretin, arginine, and other biological secretagogues and potentiators. The enhancement of beta cell function can be indicated by the presence, absence, or abnormal levels of the biomarkers.

The biomarkers used may relate to inhibition of beta cell function. Suitable biomarkers include, but are not limited to, α-hydroxybutyrate (AHB), glutamate, γ-aminobutyric acid (GABA), and other component with demonstrated beta cell toxicity or suppressor of insulin secretion in response to glucose stimulation. In a preferred embodiment, the biomarker is AHB.

The biomarkers used may relate to muscle and/or hepatic insulin resistance. Suitable biomarkers include, but are not limited to, ferritin, iron saturation, acyl-carnitine, carnitine, creatine, and a branched-chain amino acid. As understood by one skilled in the art, the term “iron saturation” refers to a biomarker measured by the amount of iron divided by the amount of transferrin in serum.

The biomarkers used may relate to total glycemic control. Suitable biomarkers include, but are not limited to, glucose, HbAlc, fructosamine, glycation gap, D-mannose, and 1,5-anhydroglucitol (1,5-AG), and, optionally, α-hydroxybutyrate (AHB).

The biomarkers used may relate to inflammation control. Suitable biomarkers include, but are not limited to, lipoprotein-associated phospholipase A2 (LpPLA2), fibrinogen, high sensitivity C-reactive protein (hsCRP), myeloperoxidase (MPO) and F2-isoprostanes and, optionally, one or more biomarkers selected from the group consisting of serum amyloid A and variants thereof; HSP-70; IL-6; TNF-α; haptoglobin and variants thereof; secretory phospholipase A2 (sPLA2); pregnancy-associated plasma protein-A (PAPP-A); and mannose binding lectin (MBL) level, activity, genetic polymorphisms or known haplotypes thereof.

In some embodiments, the biomarkers used in the method may contain at least three from the physiological classes listed above, and at least one from the remaining three classes. Alternatively, the biomarkers used in the method may contain at least three from the physiological classes listed above, and at least two from the remaining classes. Alternatively, the biomarkers used in the method may contain at least three from the physiological classes listed above, and at least three from the remaining classes. Alternatively, the biomarkers used in the method may contain at least three from the physiological classes listed above, and at least four from the remaining classes. Alternatively, the biomarkers used in the method may contain at least three from the physiological classes listed above, and at least five from the remaining classes.

The biomarkers used in the method may be from the same or different physiological classes. One or more biomarkers used may relate to more than one physiological process.

An exemplary combination of biomarkers measured in the method comprises free fatty acids, c-peptide, and adiponectin. Another exemplary combination of biomarkers measured in the method comprises free fatty acids, c-peptide, adiponectin, ferritin, and L-GPC. Another exemplary combination of biomarkers measured in the method comprises free fatty acids, c-peptide, adiponectin, ferritin, L-GPC, and AHB. Another exemplary combination of biomarkers measured in the method comprises free fatty acids (FFA), glucose, myeloperoxidase, and L-GPC. Another exemplary combination of biomarkers measured in the method comprises free fatty acids, glucose, myeloperoxidase, insulin, fibrinogen, and leptin. In another exemplary combination, the combination of biomarkers measured in the method comprises, c-peptide, AHB, myeloperoxidase, and HDLC. In another exemplary combination, the combination of biomarkers measured in the method comprises, c-peptide, FFA, myeloperoxidase, and HDLC.

The biomarkers used in the method also include those described in Table 1 below. Table 1 lists the panels of predictive and informative diagnostic analytes in 5 different metabolic processes that underpin the development of T2DM.

TABLE 1
Biomarkers in 5 different metabolic processes (note
that some analytes may inform more than one category)

| Panel | Core Biomarkers | Optional/Accessory |
|---|---|---|
| Total | glucose, HbAlc, fructosamine, | AHB |
| Glycemic | glycation gap, D-mannose, | |
| Control | 1,5 A-G | |
| Beta Cell | serum amylase, anti-GAD | GLP-1, fasting insulin, |
| Function | auto-antibody, c-peptide, | ratio c-peptide/insulin, |
| | intact pro-insulin, | ratio intact pro-insulin/ |
| | c-pep/pro-insulin, AHB | insulin, ratio [c-peptide + |
| | | pro-insulin]/insulin, |
| | | other autoantibodies |
| | | against pancreatic islet |
| | | cells such as amylase |
| | | alpha2 autoantibody, |
| | | AHB |
| Insulin | D-mannose, leptin, | Fasting insulin, oleic |
| Resistance | adiponectin, ferritin, | Acid, L-GPC, GLP-1, |
| | and Free Fatty Acids | alpha hydroxybutyrate, |
| | (FFA) | MBL amount, activity, |
| | | or genetic |
| | | polymorphisms |
| | | thereof, BMI, LP- |
| | | IR Score |
| Inflammation | LpPLA2, fibrinogen, | HSP 70, IL-6, TNF-α, |
| | hsCRP, Myeloperoxidase | SAA variants, |
| | (MPO), F2-isoprostanes | haptoglobin variants; |
| | | secretory |
| | | phospholipase A2 |
| | | (sPLA2); pregnancy- |
| | | associated plasma |
| | | peptide A (PAPP- |
| | | A), MBL amount, |
| | | activity, or genetic |
| | | polymorphisms thereof |
| Lipids and | FFA, triglycerides, RLP, | Lipid particle |
| Lipoproteins | ApoB-48, L-GPC LP-IR | measurements; |
| | score, LDL-c, HDL-c | the measurement of |
| | | cholesterol and/or |
| | | triglycerides contained |
| | | within one or more |
| | | specific subtypes of |
| | | lipoprotein particles |
| | | and remnants thereof, |
| | | and Mannose Binding |
| | | Lectin, MBL) and |
| | | associated genetic |
| | | polymorphisms and |
| | | known haplotypes |
| | | thereof |

All protein biomarkers claimed refer to any and all of the variants comprising the “wild type” protein, variants due to single nucleotide polymorphisms (SNPs), variants due to differential associations of multiple primary chains into secondary, tertiary, quaternary structures, post-translational modifications, glycosylations, fragments, dimers, trimers, tetramers, and n-mers, etc.

More descriptions about biomarkers and relating physiological processes can be found in PCT/US13/069257, entitled “Method of Determining and Managing Total Cardiodiabetes Risk,” filed Nov. 8, 2013; U.S. patent application Ser. No. 14/153,994, entitled “Method of Detection of Early Insulin Resistance and Pancreatic Beta Cell Dysfunction in Normoglycemic Patients,” filed Jan. 13, 2014; U.S. patent application Ser. No. 14/216,850, entitled “Method of Generating an Index Score for MBL Deficiency to Predict Cardiovascular Risk,” filed Mar. 14, 2014; and U.S. patent application Ser. No. 14/154,074, entitled “ Method of Detection of Clinically Significant Post-Prandial Hyperglycemia in Normoglycemic Patients,” filed Jan. 13, 2014; all of which are herein incorporated by reference in their entirety.

The biomarker measured in the method can be a protein in a form of a monomer, a multimer, a complex with one or more other organic molecules, a normal (wild-type) form, a genetic variant with altered amino acid sequence or conformation, an isoform, a glycoform, a post-translationally modified form, an oxidized form, a form with altered biological function, a fragment/product of enzymatic cleavage, or an adduct with another chemical moiety.

After obtaining measured levels of a combination of the biomarkers, the measured values are mathematically transformed by means of an algorithm to generate a score.

In some embodiments, the mathematical transformation can comprise the steps of: i) multiplying the measured level of each of the biomarkers by a pre-determined exponent; ii) multiplying the products of the exponentiation generated from step i); and iii) logarithmically transforming the multiplied product generated from step ii).

In some embodiments, the mathematical transformation can comprise the steps of: i) multiplying the measured level of each of the biomarkers by a pre-determined exponent to create a product of exponentiation for each of the biomarkers; ii) multiplying the product of the exponentiation for each of the biomarkers generated from step i) other than the product of exponentiation generated from HDLC to form a multiplied product of the exponentiations; iii) dividing the multiplied product of the exponentiations generated from step (ii) by the product of exponentiation generated from HDLC to generate a divided product; and logarithmically transforming the divided product generated from step iii).

As will be understood by one skilled in the art based on the teachings herein, the algorithm and the exponent for each biomarker in the algorithm can be determined by a variety of techniques and can vary widely. In one example of determining appropriate exponent for each biomarker, multivariable logistic regression (MLR) is performed using the biomarker values found in the patients to predict the IGT determined by a 2-hour OGTT, e.g., by fitting C-peptide area under the curve (AUC)*FFA AUC. The pre-determined exponent for each biomarker can be derived from values within the 90%, 95%, or 99% confidence interval of the biomarker measurement distribution in a population study. For instance, the pre-determined exponent for each biomarker is the median or mean from values within the 90%, 95%, or 99% confidence interval of the biomarker measurement distribution in the population study. There are several methods for variable (biomarker) selection that can be used with MLR, whereby the biomarkers not selected are eliminated from the model and the exponents for each predictive biomarker remaining in the model are determined. These exponents are then multiplied by the biomarker content of the sample (expressed as a percentage of total biomarkers in the sample) and then summed to calculate a weighted score. The resulting score can then be compared with a particular cutoff score (i.e., a threshold), above which a subject is diagnosed having high likelihood of having abnormal glucose tolerance (or increased likelihood of early insulin resistance). A statistical model that may be used to derive the algorithm and the exponent for each biomarker in the algorithm is exemplified in Example 1.

An exemplary algorithm comprises obtaining the amounts of the biomarker analytes from at least three of the physiological classes listed in step (a) above, and optionally at least one, two, three, four, or five of the remaining classes; multiplying the amount of each analyte component (biomarker) by an exponent; then multiplying each respective values of measured analyte component after the exponentiation; and taking the natural log of the multiplied product.

Another exemplary algorithm comprises obtaining the amounts of the biomarker analytes from at least three of the physiological classes listed in step (a) above, and optionally at least one, two, three, four, or five additional biomarkers; multiplying the amount of each analyte component (biomarker) by an exponent; then multiplying each respective values of measured analyte component after the exponentiation, other than the exponentiation generated from HDLC; dividing the multiplied product exponentiations (generated from the biomarkers other than HDLC) by the product of the product of the exponentiation generated from HDLC; and taking the natural log of the multiplied product.

The exponents of the respective measured component values included in the algorithm can be derived from a population study. For example, the exponents for individual components can be chosen from the range of actual measured values within the 90%, 95%, or 99% confidence interval of the distribution of values for each respective component in the population study, with particular preference for selection of the median or mean values within the 90% confidence internal of a distribution of the measurements of respective components in the population study.

Scores can be calculated based on the following exemplary algorithms, as described in Examples:

$$- 0.7 + LN [ \frac{{Cpeptide}^{1.4} * {AHB}^{1.5} * {MPO}^{0.8}}{{HDLC}^{d 2.1}} ]$$

$$2.9 + LN [ \frac{{Cpeptide}^{1.5} * {FFA}^{0.9} * {MPO}^{0.7}}{{HDLC}^{2.2}} ]$$

All biomarkers were transformed using natural logarithm, which produced the following IGT risk score:

$${IGT}_{RS 4 A} = - 0.7 + LN [ \frac{{Cpeptide}^{1.4} * {AHB}^{1.5} * {MPO}^{0.8}}{{HDLC}^{d 2.1}} ]$$

A similar model included FFA in place of AHB, i.e.

$${IGT}_{RS 4 F} = 2.9 + LN [ \frac{{Cpeptide}^{1.5} * {FFA}^{0.9} * {MPO}^{0.7}}{{HDLC}^{2.2}} ]$$

The intercept term was added to adjust for the mean prevalence of IGT in the study cohort (about 34%), and may be adjusted when the pre-test prevalence is different.

The baseline risk factors, age, gender, BMI, etc., were combined in the logistic regression model as:

RiskScore+Beta1*Age+Beta2*gender+Beta3*BMI+Beta4*LN(fasting glucose)+Beta5*Ln(fasting insulin)

A beta weight could be multiplied by the RiskScore as a ‘slope calibration’ in future examples, also an intercept could be added for ‘intercept calibration’.¹⁶

As used herein, a “control” or “reference value” is an empirical value (score) derived from a normal human subject or from a population study using the algorithms herein, depending on the biomarkers used in the algorithms. The likelihood of a subject having abnormal glucose tolerance or early insulin resistance is relative to the control score or reference value. In one embodiment, the reference value is derived from pre-defined, empirical calculation of OGTT scores based on the biomarker levels from a normal individual or population (i.e., known to have normal glucose tolerance, e.g., a 2-hour OGTT test. In one embodiment, the reference value is derived from pre-defined, empirical calculation of OGTT scores based on the biomarker levels from a population of randomly chosen subjects, with or without normal glucose tolerance. In one embodiment, when the empirical scores from a population study is used, the normal distribution of which can be used to determine the control or reference value, e.g., the score from the bottom 5%, 10%, 15%, 20%, 25%, or 50% of the population can be used as a cutoff value, i.e., a reference value. A score higher than this reference value correlates with an increased likelihood of the subject having abnormal glucose tolerance, and a score lower than this reference value correlates with a decreased likelihood of the subject having abnormal glucose tolerance.

The resulting score is correlated to the likelihood (odds ratio) of elevation of blood glucose to ≧140 mg/dL at 2 hours post oral glucose tolerance test (OGTT) and/or a mixed meal challenge.

The score generated by the algorithm is an odds ratio that corresponds to the likelihood that a patient will have a true positive IGT. The odds ratio is a measure of relative risk determined by logistic regression. When compared to a reference value from a population study, an elevated score correlates with an increased likelihood of elevation of blood glucose to >140 mg/dL at 2 hours after oral glucose tolerance test and indicates that the subject has an increased likelihood of having abnormal glucose tolerance; and a low score correlates with a decreased likelihood of elevation of blood glucose to ≧140 mg/dL at 2 hours post oral glucose tolerance test and indicates that the subject has a decreased likelihood of having abnormal glucose tolerance.

The area under receiver operating characteristic (ROC) curves (AUC, c-statistics, concordance index) can summarize the continuum of model sensitivity and specificity values into a single measure. Positive likelihood ratios combine in one number the sensitivity and specificity at the cut-point threshold by dividing the proportion of true positives by the proportion of false positives. This statistic indicates how likely it is that a case will have an abnormal test compared to a reference, given 2 random patients, one of whom is a case and the other a reference. The c-statistics (AUC) shows the accuracy and sensitivity of the prediction from a method. Typically, the method predicts the likelihood (odds ratio) of elevation of blood glucose to ≧140 mg/dL at 2 hours post OGTT with a c-statistic (AUC value in an ROC plot) of at least 0.70.

The resulting score generated by the method may be used as a proxy for a 2-hour time point blood glucose measurement, and can replace the oral glucose tolerance test (OGTT) to predict likelihood of the subject having abnormal glucose tolerance.

The method can further comprise obtaining values for one or more base model factors to predict the likelihood of the subject having abnormal glucose tolerance; calculating a based model score for the subject based on one or more values of the base model factors; and combining the score obtained from step b) (i.e., the score calculated based on the measured levels of the biomarkers via a mathematical transformation) with the calculated base model score. The combined score can then be compared to reference values from a population. Exemplary base model factors are age, sex, BMI, fasting glucose, HbAlC, and fasting insulin.

### Diabetes and Related Disease Conditions

Diabetes, or diabetes mellitus (DM), is a group of diseases characterized by high blood glucose levels that result from defects in the body's ability to produce and/or use insulin. Diabetes encompasses Type 1 diabetes and Type 2 diabetes, which are chronic conditions, and gestational diabetes, which occurs during pregnancy and may resolve itself after the baby is delivered. A precursor to Type 2 diabetes is the potentially reversible-condition prediabetes, which refers to a condition when blood sugar levels are higher than normal but not high enough to be considered diabetes. Prediabetes and Type 2 diabetes result from the body's inability to use insulin efficiently, a condition known as insulin resistance. If left untreated, insulin resistance leads to full-blown Type 2 diabetes.

The control of blood glucose levels is critical to human health. Insulin plays a central role in glucose regulation: it is the hormone that brings blood glucose into cells. Without sufficient insulin to bring glucose into the cells, blood glucose becomes elevated, the cells “starve” for glucose, and the body must use alternative pathways to produce energy for vital organs, e.g., generating ketone bodies and free fatty acids (FFAs) to fuel the brain and heart, respectively. The pancreatic beta cells normally secrete insulin in response to a meal or a “glucose load” during an oral glucose tolerance test (OGTT), thus lowering the level of blood glucose by bringing glucose into the cells of the body. This process of glucose homeostasis can be dysregulated in a number of ways, resulting in poor control of blood glucose levels. When a patient's glucose balance is dysregulated such that the patient's blood glucose level becomes higher than normal for short or long periods of time, it indicates that the patient has developed or is developing diabetes.

Type 1 diabetes (T1DM) is a type of diabetes formerly known as “early onset” diabetes because it is an acute illness usually occurring in childhood or adolescence, but becomes evident in adults. In this condition, the patient will suddenly become very sick, with high blood sugar due to rapid and catastrophic failure of the pancreas to produce enough insulin. The patient requires injections of insulin in order to maintain normal levels of blood sugar to survive. The cause is commonly understood to derive from a viral infection and/or autoimmunity. Full-blown T1DM requires that patients be treated with exogenous insulin, because patients do not make enough insulin by themselves to survive. However, there are milder forms of T1DM that progress more slowly to insulin-dependence, or in which a patient may need insulin for a short period of time, and then go off of insulin and maintain their normal blood glucose regulation.

Type 2 diabetes (T2DM) is different physiologically than T1DM. T2DM is characterized by abnormally high blood glucose and abnormally high insulin levels. Also, T2DM does not have an acute onset of symptoms like T1DM. In contrast, it develops gradually over time, usually years, and therefore was formerly known as “adult onset” diabetes. T2DM is related to diet and lifestyle factors such as eating a high-sugar, high-carb diet, lack of exercise, and development of obesity, in particular abdominal obesity. Because of the sedentary lifestyle and poor diet in the western world, there is an epidemic of T2DM in the US and Europe that parallels the rise in the number of obese and morbidly obese adults. Because more children are also becoming obese, more cases of T2DM are developing in childhood. The consequences for development of T2DM is a radical increase in the risk of cardiovascular disease, termed cardiodiabetes, such as increased risk of heart attacks, strokes, high blood pressure, atherosclerosis, coronary artery disease, and related conditions.

Insulin resistance (IR) is the earliest stage of T2DM. The development of T2DM is preceded by a substantial period of abnormal metabolism during which lifestyle and diet intervention, including weight loss, can completely prevent and reverse the development of the disease in most people. Most patients exhibit signs of metabolic syndrome, as described below. Patients with insulin resistance generally have conditions such as hyperinsulinemia, impaired glucose tolerance, dyslipidemia (hypertriglyceridemia and decreased high-density lipoprotein (HDL) cholesterol) and hypertension. Chronic inflammation may also drive the development of insulin resistance. Insulin resistance is a change in physiologic regulation such that a fixed dose of insulin causes less of an effect on glucose metabolism than it typically causes in normal individuals, i.e., blood glucose in insulin-resistance patient does not drop as much or as fast as it should in response to increases in insulin. The normal compensatory response to insulin resistance is an even higher increase in insulin secretion that results in hyperinsulinemia. If the hyperinsulinemia is sufficient to overcome the insulin resistance, glucose regulation remains normal; if not, type 2 diabetes ensues.

“Metabolic syndrome” is associated with insulin resistance. It is a cluster of metabolic abnormalities involving body fat distribution, lipid metabolism, thrombosis, blood pressure regulation, and endothelial cell function. This cluster of abnormalities is referred to as the insulin resistance syndrome or the metabolic syndrome. Eventually, blood glucose remains elevated even in the fasting state as the insulin-resistant patient progresses towards T2DM. The pancreatic beta cells wear out pumping the required insulin, and over time, the pancreatic islets (and the beta cells they contain) are damaged perhaps due to the exhaustion. The pancreatic beta cells begin to secrete more immature insulin (pro-insulin) in an attempt to keep up with the demand, and therefore, in the blood of insulin-resistant patient who is developing T2DM, biomarkers of pancreatic beta cell dysfunction such as higher levels of insulin, pro-insulin and c-peptide (Singh et al., “Surrogate markers of insulin resistance: A review,” World J Diabetes 1(2): 36-47 2010, which is hereby incorporated by reference in its entirety) can present.

The term “pre-diabetes” is essentially synonymous with insulin resistance and metabolic syndrome of Type 2 diabetes, but has specific laboratory-measured values associated with it. Doctors screen patients for diabetes if they have known risk factors, a family history of diabetes, high blood pressure, BMI greater than 25, or if they have abnormal cholesterol levels (defined as HDL-C below 35 mg/dL (0.9 mmol/L) or triglyceride level above 250 mg/dL (2.83 mmol/L).

If full-blown T2DM develops and is left undiagnosed and untreated, patients should be treated with insulin-sensitizing drugs which may help make their cells more responsive to insulin so that the pancreas does not have to work as hard. Blood glucose balance can be maintained with insulin sensitizing drugs, or maintained and/or reversed by effectuating diet and lifestyle modifications and weight loss. Unlike full-blown T1DM, T2DM may be reversible in many patients. However, if T2DM progresses far enough, the pancreatic beta cells become unable to secrete enough insulin on their own due to exhaustion, and the patients may progress to the last stage of T2DM where they cannot make enough insulin. Thus, the patients will become insulin-dependent and need exogenous insulin injection to survive, because their pancreatic beta cells no longer function. This is the worst stage of T2DM and can be fatal because while a patient can be administered exogenous insulin, their body may still be resistant to its effects. These patients are at dramatically increased risk for cardio-diabetic morbidity and mortality.

### Diagnoses of Abnormal Glucose Metabolism

Disorders of glucose metabolism on the sliding scale of T2DM are defined per the following laboratory test values:

Diabetes (or diabetes mellitus) is diagnosed clinically by demonstrating any of the following four criteria (results should be confirmed by retesting on a subsequent occasion): fasting glucose level ≧126 mg/dL; glycosylated hemoglobin (HbAlC) level ≧6.5%; 2-hour glucose level ≧200 mg/dL during glucose tolerance testing (e.g., two hours after a 75 g oral glucose load); random glucose values ≧200 mg/dL in the presence of symptoms of hyperglycemia.

Insulin resistance (IR) is diagnosed clinically according to the following laboratory analysis: a state in which higher concentrations of insulin are required to exert normal effects; blood glucose levels may be normal but fasting insulin levels may be high because of compensatory insulin secretion by the pancreas. Insulin levels can be defined for certain individuals. For instance, in some tests optimal fasting insulin level is defined as 3-9 μU/mL, intermediate insulin level is defined as >9 μU/mL and <12 μU/mL, and high insulin level is defined as ≧12 μU/mL.

“Pre-diabetes” can be diagnosed by demonstrating one of the following: the glycated hemoglobin (HbAlC) level of 6.0% to 6.5%, a fasting blood glucose level from 100 to 125 mg/dL (or 5.6 to 6.9 mmol/L), or a blood glucose value of 140 to 199 mg/dL (or 7.8 to 11.0 mmol/L) at the 2-hour time point of an OGTT. If the patient has pre-diabetes, doctors will usually check fasting blood glucose, HbAlC, total cholesterol, HDL cholesterol, low-density lipoprotein (LDL) cholesterol, and triglycerides at least once a year.

“Impaired glucose tolerance” (IGT) is assessed by typically evaluating if glucose levels are 140-199 mg/dL 2 hours after a 75 g oral glucose load. The elevation of 2-hour blood glucose value indicates a patient has an IGT.

Impaired fasting glucose is diagnosed by typically evaluating if fasting glucose levels (i.e., glucose levels after an 8-hour fasting) are 100-125 mg/dL 2 hours after a 75 g oral glucose load. The elevation of 2-hour blood glucose value indicates a patient has an IGT.

It will be appreciated by those skilled in the art of diabetes diagnostics and treatment that the patient population in which this invention has clinical utility do not fit the above clinical definitions for insulin resistance, pre-diabetes, metabolic syndrome, impaired fasting glucose, T2DM, or T1DM (insulin-dependent). This test does not detect insulin resistance or place patient on a scale between normal glucose tolerance (NGT) and diabetes, because the patient population in which the test predicts abnormal first-phase insulin response are by definition NGT with normal glucose and insulin levels at baseline and the 2 hour time point, and do not meet the definition of insulin resistance based on their lipid values on the Lipoprotein Insulin Resistance (LP-IR) score. Thus, while for the purpose of illustrating the utility of the invention the patients were split into groups by glucose tolerance and degree of insulin resistance for the purpose of analyzing data in the different groups, this is for illustrative purposes to show the utility of the test in the NGT, non-insulin resistant “normal” group. The test does not have clinical utility as an early predictor of risk once the patient has met the criteria for Impaired Glucose Tolerance (IGT) or Diabetes.

### Therapy Regimen

After the subject is determined to have an increased likelihood of having abnormal glucose tolerance or early insulin resistance, a therapy/treatment regimen can be selected based on the elevated score to prevent the subject from developing or treat the subject for diabetes or related cardio-diabetes condition and comorbidities.

Methods according to the invention may also involve administering the selected therapy regimen to the subject. Accordingly, the invention also relates to methods of treating a subject to reduce the risk of diabetes or related cardio-diabetes condition and comorbidities.

The selected therapy regimen can comprise administering drugs or supplements. The drug or supplement may be any suitable drug or supplement useful for the treatment or prevention of diabetes and related cardio-diabetes condition and comorbidities. Examples of suitable agents include an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a PCSK9 inhibitor, an MTP inhibitor, mipmercin, a calcium channel blocker, a beta-adrenergic receptor blocker, an angiotensin system inhibitor, a glitazone, a GLP-1 analog, thiazolidinedionones, biguanides, neglitinides, alpha glucosidase inhibitors, an insulin, a dipeptidyl peptidase IV inhibitor, metformin, a sulfonurea, peptidyl diabetic drugs such as pramlintide and exenatide, or combinations thereof. The agent is administered in an amount effective to treat diabetes or related cardio-diabetes condition and comorbidities, or to lower the risk of the subject developing a future diabetes or related cardiodiabetes condition and comorbidities.

The drugs and/or supplements (i.e., therapeutic agents) can be administered via any standard route of administration known in the art, including, but not limited to, parenteral (e.g., intravenous, intraarterial, intramuscular, subcutaneous injection, intrathecal), oral (e.g., dietary), topical, transmucosal, or by inhalation (e.g., intrabronchial, intranasal or oral inhalation, intranasal drops). Typically, oral administration is the preferred mode of administration.

A therapy regimen may include providing a report to a qualified healthcare provider and/or patient, or providing a referral to a healthcare specialist or related specialist based on the determined score. The reports may be related to the subject's likelihood of developing diabetes or related cardio-diabetes condition and comorbidities based on the determined score. The reports may include suggested therapy regimens selected based on the subject's diabetes or related cardio-diabetes condition and comorbidities.

The report may take the form of a written report, a verbal discussion, a faxed report, or an electronic report accessed by a computing device or hand-held smart-phone device. Comments may be added to the report that aid in data interpretation, diagnosis, and choice of therapy. This report may be transmitted or distributed to a qualified healthcare provider or directly to the patient. Following transmission or distribution of the report, the subject may be coached or counselled based on the therapy recommendations.

Qualified healthcare provider is defined as a physician (MD, DO), nurse, registered dietician, pharmacist, health consultant, or other appropriately trained individual qualified to counsel patients on health-related issues. Healthcare specialists may be a cardiologist, endocrinologist, opthamologist, lipidologist, weight loss specialist, registered dietician, “health coach”, personal trainer, etc. Further therapeutic intervention by healthcare specialists based on the determined score may take the form of cardiac catherization, stents, imaging, coronary bypass surgeries, EKG, Doppler, hormone testing and adjustments, etc.

A therapy regimen may also include giving recommendations on making or maintaining lifestyle choices useful for the treatment or prevention of diabetes or related cardiodiabetes condition and comorbidities based on the results of the score. The lifestyle choices can involve changes in diet, changes in exercise, reducing or eliminating smoking, or a combination thereof. For example, the therapy regimen may include glucose control, lipid metabolism control, weight loss control, and smoking cessation. The lifestyle choice is one that will affect risk for developing or having diabetes or related cardio-diabetes condition and comorbidities.

The recommendations may be provided by a healthcare provider. The healthcare provider can repeat an interaction with a patient after a period of time to reinforce recommendations and monitor progress.

Monitoring can also assess the risk for developing further diabetes or related cardio-diabetes condition and comorbidities. This method involves determining if the subject is at an elevated risk for developing diabetes or related cardio-diabetes condition and comorbidities, which may include assigning the subject to a risk category selected from the group consisting of high risk, intermediate risk, and low risk (i.e., optimal) groups for developing or having diabetes or related cardio-diabetes condition and comorbidities. This method also involves repeating the determining if the subject is at an elevated risk for developing diabetes or related cardio-diabetes condition and comorbidities after a period of time (e.g., before and after therapy). The method may also involve comparing the first and second risk categories obtained at different period of time, and determining, based on the comparison, if the subject's risk for developing diabetes or related cardio-diabetes condition and comorbidities has increased or decreased, thereby monitoring the risk for developing diabetes or related cardio-diabetes condition and comorbidities.

### System for Predicting Likelihood of Abnormal Glucose Tolerance or Insulin Resistance

The methods described herein may be implemented using any device capable of implementing the methods. Examples of devices that may be used include, but are not limited to, electronic computational devices, including computers of all types. When the methods are implemented on a computer, the computer program that may be used to configure the computer to carry out the steps of the methods may be contained in any computer readable medium capable of containing the computer program.

For example, the computer system can optionally comprise a module configured to obtain measured level of a combination of biomarkers from a biological sample in a subject. The computer system can optionally comprise a measuring module configured to yield detectable signal from an assay indicating the amount of each biomarker in the sample. The computer system can further optionally comprise a calculating module configured to calculate a score based on the measured levels of the biomarkers using a mathematical transformation. Optionally, the computer system can comprise a storage module configured to store output information from the calculating module. Optionally, the computer system can comprise an output module for displaying the output information from the calculating module, or generating a report from the output information for the user.

The measuring module may comprise an assay that is automated on robotic equipment.

The calculating module may comprise a software to automate the calculation of the score. The calculating module may also comprise a software to calculate pre-determined parameters (e.g., exponents of the biomarkers used in the algorithm).

The computer program, including the reference levels of the biomarkers and base model factors, and pre-determined parameters (e.g., exponents of the biomarkers used in the algorithm) may be contained in a computer readable medium. Examples of computer readable medium that may be used include but are not limited to diskettes, CD-ROMs, DVDs, ROM, RAM, and other memory and computer storage devices.

The computer system that may be used to configure the computer to carry out the steps of the methods may also be provided over an electronic network, for example, over the internet, world wide web, an intranet, or other network. It can also be downloaded to a computer or other electronic device such as a laptop, smart-phone, tablet, or the IT network in a provider's office. An exemplary application that carries out the steps of the methods downloadable to a computer or a smart-phone has been described in details in U.S. patent application Ser. No. 14/144,269, entitled “An Interactive Web-based Platform for Facilitating Biomarker Education and Patient Treatment Analysis,” filed Dec. 30, 2013; which is herein incorporated by reference in its entirety.

### EXAMPLES

### Example 1

### Detection of Impaired Glucose Tolerance Fasting Blood Samples Using Modified Index Scores

Participants from the Insulin Resistance Atherosclerosis Study¹¹ (IRAS) (n=167), ACTos NOW¹² (n=142), and Health Diagnostic Laboratory (HDL) metabolic health study¹³ (n=441) cohorts who underwent an OGTT and had at least 250 μL of fasting plasma blood sample available at HDL were included in this study. Subjects were protected under IRB approval or under clinical HIPAA authorization, depending on whether samples were collected under study protocol or in a routine clinical setting.

A cross-sectional study of 750 participants combined from IRAS, ACT NOW, and HDL cohorts were randomly split into training and validation groups based on IGT status. Multivariable logistic regression was used on the training group data with Schwarz Bayesian Criteria (SBC) minimization to identify the best subset of biomarkers associated with IGT. Two novel composite IGT risk scores were developed from a set of 15 log-transformed candidate blood biomarkers. Participants in the validation group were then scored, and the statistical association, discrimination, calibration, and reclassification performance were tested.^{14,15}. The IGT risk score was tested as a continuous variable per a one standard deviation (SD) increase, and as a categorical variable by quartiles. To test whether the risk score performed uniquely in a subpopulation, interactions were added between the IGT risk score with age, gender, and BMI, and a Likelihood Ratio Test was performed. The participants' median age and BMI were 51 years and 30 kg/m², respectively; 63% were female (Table 2). The study population was racially diverse as 61% reported white, 26% Hispanic, and 12% black. The median lipid and glucose values were normal; however, about 46% had IGT and/or IFG and 5% were diabetic.

TABLE 2
Oral Glucose Tolerance Test Patients' Characteristics

| | Overall | | Training | | Validation | |
|---|---|---|---|---|---|---|
| Variable | (N = 750) | | (N = 374) | | (N = 376) | |
| Age [years] | 51 | (40, 59) | 52 | (40, 60) | 50 | (40, 59) |
| Male | 275 | (37%) | 132 | (35%) | 143 | (38%) |
| Race/Ethnicity†: n (%) | | | | | | |
| Black | 37 | (12%) | 17 | (10%) | 20 | (14%) |
| Hispanic | 79 | (26%) | 49 | (29%) | 30 | (22%) |
| Other | 6 | (2%) | 3 | (2%) | 3 | (2%) |
| White | 187 | (61%) | 101 | (59%) | 86 | (62%) |
| Cohort: n (%) | | | | | | |
| HDL | 441 | (59%) | 204 | (55%) | 237 | (63%) |
| IRAS | 167 | (22%) | 97 | (26%) | 70 | (19%) |
| ACTNOW | 142 | (19%) | 73 | (20%) | 69 | (18%) |
| BMI [kg/m²] | 30 | (26, 35) | 30 | (26, 35) | 30 | (27, 36) |
| Diastolic BP^{†} [in Hg] | 74 | (69, 81) | 75 | (69, 80) | 73 | (67, 81) |
| Systolic BP] ^{†} [in Hg] | 121 | (110, 134) | 121 | (109, 134) | 119 | (111, 132) |
| Glucose Status: n (%) | | | | | | |
| Normal | 365 | (49%) | 184 | (49%) | 181 | (48%) |
| IFG | 88 | (12%) | 44 | (12%) | 44 | (12%) |
| IGT | 101 | (13%) | 50 | (13%) | 51 | (14%) |
| IFG + IGT | 157 | (21%) | 81 | (22%) | 76 | (20%) |
| Diabetic | 39 | (5%) | 15 | (4%) | 24 | (6%) |
| Glucose 2-hour [mg/dL] | 120 | (93, 160) | 119 | (92, 157) | 121 | (93, 164) |
| Fasting Glucose [mg/dL] | 95 | (87, 104) | 95 | (87, 104) | 96 | (87, 104) |
| Fasting Insulin [uU/mL] | 11 | (7, 17) | 11 | (7, 17) | 11 | (7, 17) |

Candidate Blood Biomarkers

| | | | | | | |
|---|---|---|---|---|---|---|
| HDL-C [mg/dL] | 47 | (39, 58) | 47 | (39, 57) | 48 | (39, 60) |
| LDL-C [mg/dL] | 111 | (86, 137) | 112 | (84, 137) | 110 | (86, 138) |
| Total-C [mg/dL] | 181 | (152, 209) | 181 | (149, 208) | 182 | (155, 210) |
| Non-HDL-C [mg/dL] | 131 | (102, 158) | 132 | (101, 158) | 130 | (104, 157) |
| Triglycerides [mg/dL] | 105 | (74, 154) | 105 | (73, 153) | 105 | (76, 154) |
| Triglycerides/HDL Ratio | 2.2 | (1.4, 3.6) | 2.2 | (1.4, 3.5) | 2.1 | (1.4, 3.7) |
| Free Fatty Acids (FFA) | 0.56 | (0.44, 0.73) | 0.58 | (0.44, 0.75) | 0.56 | (0.44, 0.72) |
| [mmol/L] | | | | | | |
| Leptin [ng/mL] | 25 | (12, 52) | 25 | (13, 50) | 26 | (12, 56) |
| Adiponectin [ug/mL] | 9.0 | (6.0, 13.0) | 9.0 | (6.0, 13.0) | 9.0 | (7.0, 13.0) |
| Ferritin [ng/mL] | 75 | (36, 155) | 73 | (36, 154) | 76 | (36, 157) |
| Alpha-hydroxybutyrate (AHB) | 4.5 | (3.4, 6.0) | 4.6 | (3.5, 6.0) | 4.4 | (3.4, 6.0) |
| [ug/mL] | | | | | | |
| Oleic FFA [ug/mL] | 48 | (28, 69) | 47 | (25, 67) | 49 | (30, 70) |
| L-GPC [ug/mL] | 16 | (13, 23) | 16 | (12, 23) | 16 | (13, 23) |
| C-Peptide [ng/mL] | 2.7 | (2.0, 3.8) | 2.7 | (1.9, 3.8) | 2.8 | (2.0, 3.8) |
| Myeloperoxidase (MPO) | 272 | (220, 357) | 271 | (219, 354) | 277 | (222, 362) |
| [pmol/L] | | | | | | |

† The HDL cohort was missing race and blood pressure data.

The best-fitting and most parsimonious model included C-peptide, AHB, MPO, and HDL-C.

$$LN [ \frac{{Cpeptide}^{a} * {AHB}^{b} * {MPO}^{c}}{{HDLC}^{d}} ]$$

However, there was an inverse correlation with age, and possibly higher levels in men (no effect with BMI) that resulted in a significant overall interaction between the risk score and these demographics, X²(3)=12.51 (p=0.0058).

Therefore, another risk score was created where AHB was replaced by free fatty acids (FFA), which was shown to be the best substitute from the list of candidate biomarkers by SBC minimization criteria. The same set of interactions was tested between the modified risk score with age, gender, and BMI.

$$LN [ \frac{{Cpeptide}^{a} * {FFA}^{b} * {MPO}^{c}}{{HDLC}^{d}} ]$$

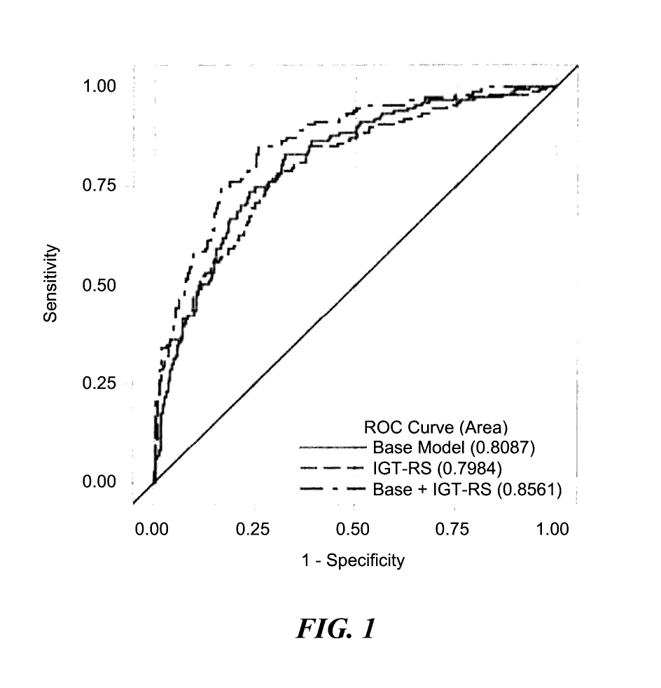

There was a significant increase in the area under the ROC curve (c-stat=0.047, p=0.0021) when the modified risk score was added to age, gender, BMI, and fasting glucose (FIG. 1).

TABLE 3
Logistic Regression Model Performance Measures in Validation
Cohort (N = 376) for Detecting Impaired Glucose Tolerance

| | | | | Hosmer- | Discrimination | | | | | |
|---|---|---|---|---|---|---|---|---|---|---|
| | | | | Lameshow | Overall | | | | | |
| | Odds | 95% CI | | Calibration | c-state | Diff. | | | | |
| Model | Ratio | Lower | Upper | P-value | (AUC) | c-stat | P-value | Sens. | Spec. | PLF |

Risk Score included MPO, HDL-C, C-peptide, and AHB

| | | | | | | | | | | |
|---|---|---|---|---|---|---|---|---|---|---|
| 1 | 2.55 | 2.08 | 3.12 | 0.091 | 0.811 | — | — | 58.5 | 82.5 | 3.3 |
| 2 | 2.78 | 2.23 | 3.46 | 0.50 | 0.825 | 0.145 | <0.0001 | 61.9 | 84.3 | 3.9 |
| 3 | 2.32 | 1.84 | 2.94 | 0.16 | 0.865 | 0.056 | 0.0003 | 69.4 | 84.7 | 4.5 |
| 4 | 2.89 | 2.21 | 3.77 | 0.69 | 0.879 | 0.062 | 0.0001 | 73.5 | 86.9 | 5.6 |

Modified Risk Score included MPO, HDL-C, C-peptide, and FFA

| | | | | | | | | | | |
|---|---|---|---|---|---|---|---|---|---|---|
| 1 | 2.67 | 2.15 | 3.31 | 0.65 | 0.798 | — | — | 55.8 | 84.3 | 3.6 |
| 2 | 3.03 | 2.38 | 3.87 | 0.26 | 0.814 | 0.134 | <0.0001 | 61.9 | 82.1 | 3.5 |
| 3 | 2.59 | 1.84 | 3.65 | 0.52 | 0.856 | 0.047 | 0.0021 | 63.9 | 84.7 | 4.2 |
| 4 | 3.37 | 2.47 | 4.60 | 0.94 | 0.875 | 0.059 | 0.0004 | 70.7 | 87.3 | 5.6 |

| Integrated Discrimination Improvement (IDI) | | | | | | Net Reclassification Improvement (NRI) | | | | |
|---|---|---|---|---|---|---|---|---|---|---|
| | Diff | P- | | P- | Overall | | | % No | P- | Avg. |
| Model | Sens. | value | Diff Spec. | value | IDI | % IGT | P-value | IGT | value | NRI |

Risk Score included MPO, HDL-C, C-peptide, and AHB

| | | | | | | | | | | |
|---|---|---|---|---|---|---|---|---|---|---|
| 2 | 0.134 | <0.0001 | 0.086 | <0.0001 | 0.220 | 46% | <0.0001 | 41% | <0.0001 | 44% |
| 3 | 0.075 | <0.0001 | 0.048 | <0.0001 | 0.124 | 40% | <0.0001 | 39% | <0.0001 | 40% |
| 4 | 0.093 | <0.0001 | 0.060 | <0.0001 | 0.153 | 47% | <0.0001 | 49% | <0.0001 | 48% |

Modified Risk Score included MPO, HDL-C, C-peptide, and FFA

| | | | | | | | | | | |
|---|---|---|---|---|---|---|---|---|---|---|
| 2 | 0.123 | <0.0001 | 0.079 | <0.0001 | 0.202 | 44% | <0.0001 | 46% | <0.0001 | 45% |
| 3 | 0.064 | <0.0001 | 0.041 | <0.0001 | 0.105 | 33% | <0.0001 | 35% | <0.0001 | 34% |
| 4 | 0.088 | <0.0001 | 0.057 | <0.0001 | 0.145 | 46% | <0.0001 | 44% | <0.0001 | 45% |

Model 1: Score
Model 2: Score + Age, Gender, BMI
Model 3: Score + Age, Gender, BMI, Log(Fasting Glucose)
Model 4: Score + Age, Gender, BMI, Log(Fasting Glucose), Log(Fasting Insulin)

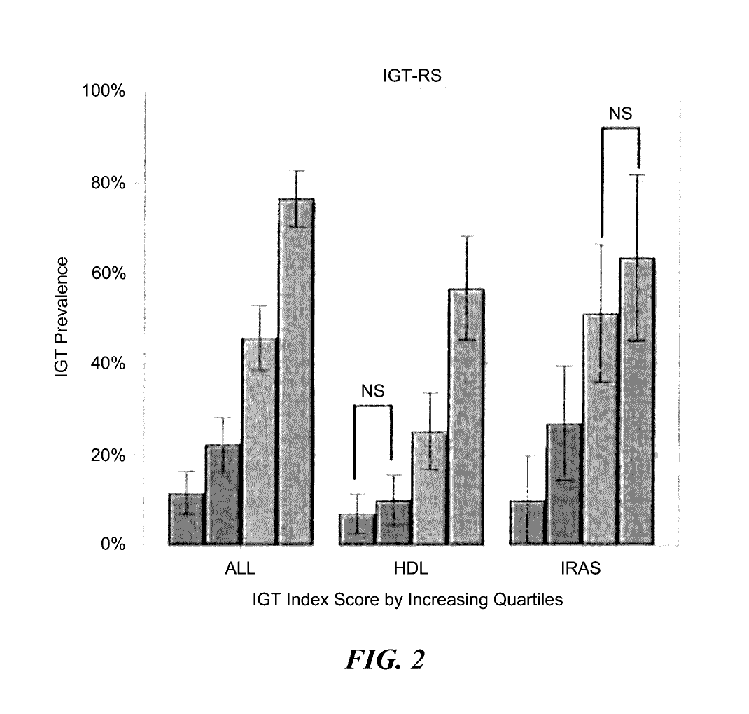

As a cohort sensitivity analysis, the modified IGT risk score was stratified by quartiles for all participants, and then the IGT prevalence rates were tested within the HDL and IRAS cohorts (FIG. 2).

Example 2

### Modified Index Score Models Incorporating Fatty Acid Analytes

A case-control study to classify impaired glucose tolerance (IGT) patients, i.e. 2-hour glucose ≧140 mg/dL, from non-IGT patients was performed using a set of fasting plasma biomarkers (primary), and a set of non-fasting (i.e. random) plasma biomarkers (secondary).

Subjects: ACT NOW (n˜140) and IRAS (n˜170) patients with at least 250 uL of plasma, and HDL (n˜440) cohorts were combined to insure sufficient power. Then the data was randomly split in half for training and validation cohorts.

TABLE 4
Demographics of ActNow, HDL, IRAS
and University of Utah subjects

| | Training | | Validation | |
|---|---|---|---|---|
| Variable | (N = 374) | | (N = 376) | |
| Age [years] | 52 | (40, 60) | 50 | (40, 59) |
| Male | 132 | (35%) | 143 | (38%) |
| Race/Ethnicity^{†}: n (%) | | | | |
| Black | 17 | (10%) | 20 | (14%) |
| Hispanic | 49 | (29%) | 30 | (22%) |
| Other | 3 | (2%) | 3 | (2%) |
| White | 101 | (59%) | 86 | (62%) |
| Cohort: n (%) | | | | |
| HDL | 204 | (55%) | 237 | (63%) |
| IRAS | 97 | (26%) | 70 | (19%) |
| ACTNOW Pioglitazone | 37 | (10%) | 41 | (11%) |
| ACTNOW Placebo | 36 | (10%) | 28 | (7%) |
| BMI [kg/m²] | 30 | (26, 35) | 30 | (27, 36) |
| Diastolic BPI† [in Hg] | 75 | (69, 80) | 73 | (67, 81) |
| Systolic BP] † [in Hg] | 121 | (109, 134) | 119 | (111, 132) |
| Impaired Glucose Tolerance: n (%) | | | | |
| Normal | 228 | (61%) | 229 | (61%) |
| IGT | 132 | (35%) | 130 | (35%) |
| Diabetic | 14 | (4%) | 17 | (5%) |

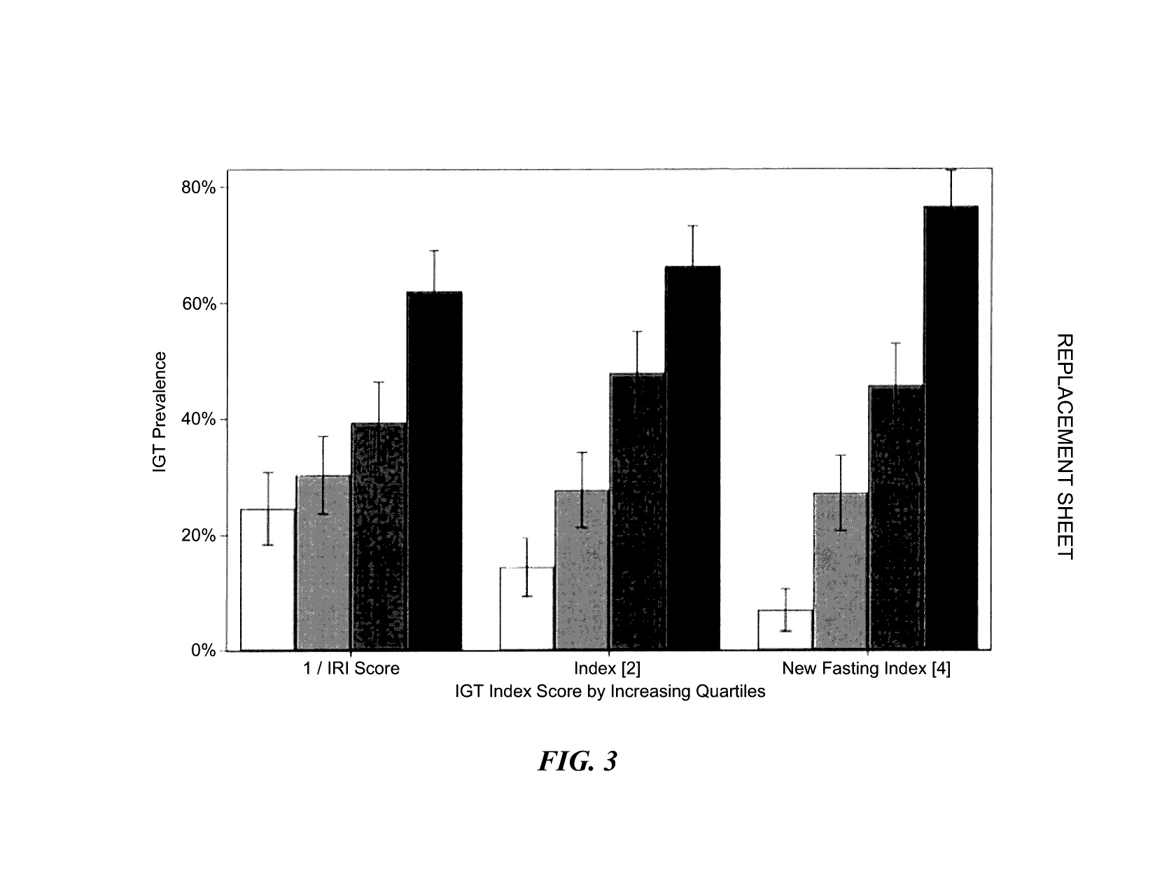

IGT prevalence by increasing quartiles were determined using three different models as shown in FIG. 3 (Model 1: 1/IRI Score; Model 2: Index [2]; Model 3: New Fasting Index [4]).

$$\begin{matrix}38.41 * [ \frac{{LGPC}^{0.184}}{{Insulin}^{0.398} {AHB}^{0.301} * {OA}^{0.126}} ] & Model 1 \\ LN [ \frac{{FFA}^{a} * {Cpeptide}^{b} * {AHB}^{c} * {Ferritin}^{d}}{{LGPC}^{e} * {Adiponectin}^{f}} ] & Model 2 \\ LN [ \frac{{Cpeptide}^{a} * {AHB}^{b} * {MPO}^{c}}{{HDLC}^{d}} ] & Model 3\end{matrix}$$

TABLE 5
Relating to the Models Represented in FIG. 3

| | | | | | | Linear Trend |
|---|---|---|---|---|---|---|
| Index | 1^{st} Quartile | 2^{nd} Quartile | 3^{rd} Quartile | 4^{th} Quartile | Overall P-value | P-value |
| 1/IRI Score | 1.0 Ref. | 1.3 | 2.0 | 5.0 | <0.0001 | <0.0001 |
| | | (0.8, 2.1) | (1.3, 7.8) | (3.2, 7.8) | | |
| Original Index | 1.0 Ref. | 2.3 | 5.4 | 12 | <0.0001 | <0.0001 |
| [2] | | (1.3, 3.8) | (3.3, 9.0) | (7.1, 19) | | |
| New Fasting | 1.0 Ref. | 5.0 | 11 | 43 | <0.0001 | <0.0001 |
| Index [4] | | (2.6, 9.5) | (6.0, 21) | (23, 84) | | |

TABLE 6
Corresponding to the Models Represented in FIG. 3

| | IDI − Average increase in | | | Average Continuous Net | | |
|---|---|---|---|---|---|---|
| | sensitivity + specificity | | | Reclassification Improvement | | |
| | | Original | Fasting | | Original | Fasting |
| Model | IRI Score | Index | Index | IRI Score | Index | Index |

Training

| | | | | | | |
|---|---|---|---|---|---|---|
| 2b | 3.4 | 10.7 | 23.1 | 22% | 30% | 42% |
| 3b | 0.0* | 3.9 | 11.0 | 10% | 26% | 36% |
| 4b | 1.4* | 5.4 | 13.5 | 23% | 23% | 37% |

Validation

| | | | | | | |
|---|---|---|---|---|---|---|
| 2b | 0.1* | 6.9 | 24.3 | 6% | 29% | 47% |
| 3b | 1.8* | 2.0 [symbol] | 14.6 | 0%* | 18%† | 39% |
| 4b | 1.2* | 4.8 | 16.9 | 15%† | 27% | 45% |

Model 2b: Age, Male, BMI, Score
Model 3b Age, Male, BMI, Log(Fasting Glucose), Score
Model 4b Age, Male, BMI, Log(Fasting Glucose), Log(Fasting Insulin), Score
P-value <0.001 unless otherwise indicated († p < 0.05, * p > 0.05)

The results herein represent a novel abnormal glucose tolerance risk score which is based on measurements of a small subset of fasting biomarkers related to cardio-metabolic risk. This risk score strongly distinguished individuals with high probability for IGT, based on 2-hr 75 g OGTT. Importantly, the risk score also indicates IGT specifically and this novel blood biomarker risk score can be used by busy clinicians in order to efficiently address and intervene on IGT and risk for disease progression.

### REFERENCES

1. American Diabetes A. Standards of medical care in diabetes—2015. Diabetes Care. 2015; 38 Suppl 1:S1-93
2. Abdul-Ghani M A, et al. Plasma glucose concentration and prediction of future risk of type 2 diabetes. Diabetes Care. 2009; 32 Suppl 2:S194-S198.
3. Webb D R, et al. Screening for diabetes using an oral glucose tolerance test within a western multi-ethnic population identifies modifiable cardiovascular risk: the ADDITION-Leicester study. Diabetologica. 2011; 54(9):2237-2246.
4. DeFronzo R A, et al. Pioglitazone for diabetes prevention in impaired glucose tolerance. N Engl J Med. 2011; 364:1104-1115
5. Lindstrom J, et al. Finnish Diabetes Prevention Study. Improved lifestyle and decreased diabetes risk over 13 years . . . Diabetologia. 2013; 56:284-293
6. Knowler W C, et al. Diabetes Prevention Program Research Group. Reduction in the incidence of type 2 diabetes with lifestyle intervention or metformin. N Engl J Med. 2002; 346:393-403
7. Tuomilehto J, et al. Finnish Diabetes Prevention Study Group. Prevention of type 2 diabetes by changes in lifestyle among subjects with impaired glucose tolerance. N Engl J Med. 2001; 344:1343-1350
8. Gillies C L, et al. Pharmacological and lifestyle interventions to prevent or delay type 2 diabetes in people with impaired glucose tolerance: systematic review and meta-analysis. BMJ. 2007; 334:299
9. James C, et al. Implications of alternative definitions of prediabetes for prevalence in U.S. Adults. Diabetes Care. 2011; 34:387-391
10. Centers for Disease Control and Prevention. National Diabetes Statistics Report: Estimates of Diabetes and Its Burden in the United States, 2014. Atlanta, Ga.: US Department of Health and Human Services; 2014.
11. Wagenknecht L E, et al. The insulin resistance atherosclerosis study (IRAS) objectives, design, and recruitment results. Ann Epidemiol. 1995; 5:464-472
12. Defronzo R A, et al. Actos now for the prevention of diabetes (act now) study. BMC endocrine disorders. 2009; 9:17
13. Varvel S A, et al. Serum alpha-hydroxybutyrate predicts elevated 1 h glucose levels and early phase beta cell dysfunction during OGTT. BMJ open diabetes research & care. 2014; 2:e000038
14. Hlatky M A, et al. Criteria for evaluation of novel markers of cardiovascular risk: a scientific statement from the American Heart Association. Circulation. 2009; 119:24082416
15. Herder C, et al. The potential of novel biomarkers to improve risk prediction of type 2 diabetes. Diabetologia. 2014; 57:16-29
16. Janssen, K et al. Updating methods improved the performance of a clinical prediction model in new patients. J Clin. Epid. 2008; 61: 76-86

I/We claim:

1. A method for predicting likelihood of a subject having abnormal glucose tolerance, comprising:
a. obtaining, from a biological sample collected from a subject, measured levels of a combination of biomarkers comprising C-peptide, myeloperoxidase (MPO), and high-density lipoprotein (HDL) cholesterol (HDL-C); and
b. calculating an index score based on the measured levels of the biomarkers, wherein the index score involves a mathematical transformation;
c. wherein an elevated index score correlates with an increased likelihood of elevation of blood glucose to ≧140 mg/dL at 2 hours after oral glucose tolerance test and indicates that the subject has an increased likelihood of having abnormal glucose tolerance, and wherein a low index score correlates with a decreased likelihood of elevation of blood glucose to ≧140 mg/dL at 2 hours post oral glucose tolerance test and indicates that the subject has a decreased likelihood of having abnormal glucose tolerance.

2. The method of claim 1, wherein the likelihood can be predicted by a single measurement of a single biological sample collected from a fasting subject.

3. The method of claim 1, wherein the likelihood can be predicted by a single measurement of a single biological sample collected from a non-fasting subject.

4. The method of claim 1, wherein the index score replaces the oral glucose tolerance test (OGTT) to predict likelihood of the subject having abnormal glucose tolerance.

5. The method of claim 1, wherein the biomarkers in step (a) additionally comprise alpha hydroxybutyrate (AHB).

6. The method of claim 1, wherein the biomarkers in step (a) additionally comprise at least one biomarker relating to adipose tissue insulin resistance, and wherein the biomarker relating to adipose tissue insulin resistance is a total free fatty acid or a component fatty acid species of a total free fatty acid.

7. The method of claim 1, wherein the biomarkers in step (a) additionally comprise at least one biomarker relating to pancreatic beta cell dysfunction and/or exhaustion, and wherein the biomarker relating to pancreatic beta cell dysfunction and/or exhaustion is selected from the group consisting of intact pro-insulin, and a fragment of any form of insulin.

8. The method of claim 1, wherein the biomarkers in step (a) additionally comprise at least one biomarker relating to adipokine function, and wherein the biomarker relating to adipokine function is selected from the group consisting of adiponectin, leptin, tumor necrosis factor alpha (TNFα), resistin, visfatin, dipeptidyl peptidase-4 (DPP-IV), omentin, and apelin.

9. The method of claim 1, wherein the biomarkers in step (a) additionally comprise at least one biomarker relating to functional enhancement of insulin secretion by beta cells, and wherein the biomarker relating to functional enhancement of insulin secretion by beta cells is selected from the group consisting of linoleoyl-glycerophosphocholine (L-GPC), an incretin, arginine, and other biological secretagogues and potentiators.

10. The method of claim 1, wherein the biomarkers in step (a) additionally comprise at least one biomarker relating to inhibition of beta cell function, and wherein the biomarker relating to inhibition of beta cell function is selected from the group consisting of glutamate, gamma-Aminobutyric acid (GABA), and other biomarkers with demonstrated beta cell toxicity or suppression of insulin secretion in response to glucose stimulation.

11. The method of claim 1, wherein the biomarkers in step (a) additionally comprise at least one biomarker relating to muscle and/or hepatic insulin resistance, and wherein the biomarker relating to muscle and/or hepatic insulin resistance is selected from the group consisting of ferritin, iron saturation, acyl-carnitine, carnitine, creatine, and a branched-chain amino acid.

12. The method of claim 1, wherein the biomarkers in step (a) additionally comprise at least one biomarker relating to total glycemic control, and wherein the biomarker relating to total glycemic control is selected from the group consisting of glucose, HbAlc, fructosamine, glycation gap, D-mannose, and 1,5-anhydroglucitol (1,5-AG).

13. The method of claim 1, wherein the biomarkers in step (a) additionally comprise at least one biomarker relating to inflammation control, and wherein the biomarker relating to inflammation control is selected from the group consisting of lipoprotein-associated phospholipase A2 (LpPLA2), fibrinogen, high sensitivity C-reactive protein (hsCRP), F2-isoprostanes, serum amyloid A and variants thereof, HSP-70, IL-6, TNF-α, haptoglobin and variants thereof, secretory phospholipase A2 (sPLA2), pregnancy-associated plasma protein-A (PAPP-A), and mannose binding lectin (MBL) level, activity, genetic polymorphisms or known haplotypes thereof.

14. The method of claim 1, wherein the mathematical transformation comprises:
i. multiplying the measured level of each of the biomarkers by a pre-determined exponent to create a product of exponentiation for each of the biomarkers;
ii. multiplying the product of the exponentiation for each of the biomarkers generated from step i) other than the product of exponentiation generated from HDLC to form a multiplied product of the exponentiations;
iii. dividing the multiplied product of the exponentiations generated from step (ii) by the product of exponentiation generated from HDLC to generate a divided product; and
iv. logarithmically transforming the divided product generated from step iii).

15. The method of claim 14, wherein the pre-determined exponent for each biomarker is derived from values within the 90% confidence interval of the biomarker measurement distribution in a population study.

16. The method of claim 15, wherein the pre-determined exponent for each biomarker is the median or mean from values within the 99% confidence interval of the biomarker measurement distribution in the population study.

17. The method of claim 1, wherein the index score comprises a calculation of the form:
$$LN [ \frac{{Cpeptide}^{a} * {AHB}^{b} * {MPO}^{c}}{{HDLC}^{d}} ]$$

18. The method of claim 1, wherein the index score comprises a calculation of the form:
$$- 0.7 + LN [ \frac{{Cpeptide}^{1.4} * {AHB}^{1.5} * {MPO}^{0.8}}{{HDLC}^{d 2.1}} ]$$

19. The method of claim 1 wherein the index score comprises a calculation of the form:
$$LN [ \frac{{Cpeptide}^{a} * {FFA}^{b} * {MPO}^{c}}{{HDLC}^{d}} ]$$

20. The method of claim 1 wherein the score comprises a calculation of the form:
$$2.9 + LN [ \frac{{Cpeptide}^{1.5} * {FFA}^{0.9} * {MPO}^{0.7}}{{HDLC}^{2.2}} ]$$

21. The method of claim 1, further comprising:
a. obtaining values for one or more base model factors to predict the likelihood of the subject having abnormal glucose tolerance;
b. calculating a based model score for the subject based on one or more values of the base model factors; and
c. combining the index score obtained from step b) of claim 1 with the calculated base model score, wherein the combined score is compared to reference values from a population.

22. The method of claim 21, wherein the based model factor is fasting glucose, and wherein the base model score is calculated by logarithmically transforming a measured level of fasting glucose in the subject and multiplied by a weighting factor (beta).

23. The method of claim 21, wherein the combining the index score obtained from step b) of claim 1 with the base model score provides an improved likelihood prediction than the base model score alone.

24. The method of claim 21, wherein the combining the index score obtained from step b) of claim 1 with the base model score causes a net reclassification improvement (NRI) of the subject from having normal glucose tolerance (NGT) to impaired glucose tolerance (IGT), or from having IGT to NGT.

25. The method of claim 24, wherein the NM is at least about 10%.

26. The method of claim 1, further comprising administering a therapy regimen for the treatment or prevention of abnormal glucose tolerance.

27. The method of claim 1, further comprising monitoring the levels of the biomarkers in the subject to assess progression, improvement, normalization, and/or treatment efficacy, wherein the monitoring step comprises repeating steps a)-c) based on the levels of the biomarkers from a biological sample in the subject obtained at a later time.
